(19) United States
(12) Patent Application Publication
(10) Pub. No.: US 2016/0001015 A1
(43) Pub. Date: Jan. 7, 2016

(54) CLOSURE ELEMENT, IN PARTICULAR A SYRINGE CLOSURE ELEMENT FOR CLOSING A DISTAL OPENING OF A SYRINGE BODY IN A SEALING MANNER

(71) Applicant: Schott Schweiz AG, St. Gallen (CH)

(72) Inventors: Mustafa Kücük, Staad (CH); Christoph Huber, Kreuzlingen (CH); Levent Kusogullari, Winterthur (CH); Bastian Fischer, St. Gallen (CH); Katharina Jenöfi, St. Gallen (CH); Qazim Fetoshi, St. Gallen (CH)

(73) Assignee: SCHOTT SCHWEIZ AG, St. Gallen (CH)

(21) Appl. No.: 14/731,995

(22) Filed: Jun. 5, 2015

Related U.S. Application Data
(63) Continuation of application No. PCT/EP2013/001688, filed on Jun. 10, 2013.

(30) Foreign Application Priority Data
Dec. 7, 2012 (EP) PCT/EP2012/005063

(51) Int. Cl. A61M 5/50; A61M 5/32

(52) U.S. Cl. CPC A61M 5/504; A61M 5/3213

(57) ABSTRACT

A closure piece for closing a distal opening of a syringe cone of a syringe body includes a fastening element which is or can be arranged on the syringe body about the distal opening, and a closure cap which closes the distal opening in a sealed manner and which is connected releasably to the fastening element. The closure cap, in the area of the fastening element, is connected by material bonding to the fastening element by at least one connection point or one connection surface, and the closure cap includes a hard area and an elastomer part. The elastomer part securely closes the distal opening by being placed on or enclosing a short portion in an area of a tip of the distal opening.

### CROSS REFERENCE TO RELATED APPLICATIONS

This is a continuation of PCT application No. PCT/EP2013/001688, entitled “CLOSURE PIECE, IN PARTICULAR A SYRINGE CLOSURE PIECE FOR CLOSING A DISTAL OPENING OF A SYRINGE BODY IN A SEALED MANNER”, filed Jun. 10, 2013, which is incorporated herein by reference.

### BACKGROUND OF THE INVENTION

1. Field of the Invention

The invention relates to a closure element, in particular a syringe closure element for closing in a sealing manner a distal opening such as a syringe cone on a syringe body.

2. Description of the Related Art

Syringe closure elements of different types are known from a multitude of patent specifications. U.S. Pat. No. 6,190,364, for example, describes a syringe closure element for a syringe body. The syringe body itself comprises a distal syringe and a distal opening extending through same, in particular in the embodiment of a syringe cone so that a liquid which is located inside the syringe body can leave the syringe body through the distal opening or, respectively, the syringe cone. The syringe body moreover comprises a fastening element which is arranged or can be arranged on the syringe body. This fastening element can be part of a single component syringe or may be placed on the distal tip of the syringe body and be firmly connected with same. The fastening element moreover has threads or bayonet structures which interact with the threads of a two-component closure cap in order to hold the closure cap on the fastening element. The closure cap surrounds the region of the distal syringe or, respectively, the syringe cone and closes and seals the distal opening of the syringe cone. To be able to inject the syringe liquid, the closure cap in U.S. Pat. No. 6,190,364 is unscrewed from the fastening element and an injection cannula is secured on the syringe body in such a manner that a needle opening extending through the injection cannula forms a flow connection with the distal opening of the injection body.

One problem with syringe closure elements of this type is that it must be ensured that they cannot be reused in certain fields of applications, for example, with another medication or for another application. Such second use would present a high risk of infection of the patient due to contamination, for example though foreign particles and germs. An additional risk exists in that when reusing the syringe closure element on a syringe body other than the original one, a contamination of the second medication occurs since traces of the original medication may be present in the closure cap which could be transferred into the second medication and could lead to incorrect treatment of the patient.

To eliminate possible manipulations and a second use of the syringe closure element according to U.S. Pat. No. 6,190,364, the syringe closure element is provided with a sealing strip which is detachably connected with the fastening element as well as with the closure cap so that the sealing strip tears when the closure cap is released from the fastening element. With a closure element according to U.S. Pat. No. 6,190,364 it is disadvantageous that several separate parts must be manufactured which are subsequently connected with each other, incurring additional costs.

A syringe closure element for sealing and closing has therefore become known from EP 1600190 A1 which on the one hand recognizes manipulations at the syringe opening, and whereby on the other hand manufacture has been significantly simplified compared to U.S. Pat. No. 6,190,364. For this purpose, the fastening device in EP 1 600 190 A1 was provided with a locking device which locks with a locking device of the closure cap. Moreover the locking devices of the fastening element as well as that of the closure cap are designed such that they cannot be unlocked without damaging them. This means that after unlocking, a damage free relocking is not possible. Even though damage-free relocking is ruled out in EP 1 600 190 A1 it is possible to again lock the closure cap with the fastening element by accepting damage. Protection from multi-use—in order to ensure single use for a syringe—is also not provided by EP 1 600 190 A1.

A closure element for a pre-fillable single use plastic syringe having a syringe body and a syringe needle which is firmly connected with said syringe body's delivery end, consisting of a rigid outer plastic protective cap for the needle, consisting of a hard elastic material which can be placed directly onto the syringe body, surrounding the syringe needle and which, on the inside at least in the region of the needle tip is provided with a sealing lining consisting of a soft elastic material has become known from EP 1101505, wherein the plastic protective cap for the needle is welded or glued directly circumferentially with the syringe body whereby an intended separation point is provided in the welded or glued seam.

A closure cap that is connected with a fastening element for closing a distal opening of a syringe body is known from US 2001/003150 A. With this arrangement, it was disadvantageous that multiple use of the closure cap could not be ruled out with certainty.

EP 1 034 810 A1 shows a syringe for medical purposes with a needle connecting piece that is attached on the syringe cylinder and closed by a closure cap, for example a tip-cap, until the syringe is used, wherein said connecting piece is arranged in the form of a Luer-cone for attachment of a cannula, wherein the closure cap together with the end of the needle connecting piece is enclosed by a safety cap and the closure cap is held in the safety cap by interlocking or frictional connection, also with a retaining ring which is lockable on the needle connecting piece in its position on the needle connecting piece.

When using the syringe the connection between the safety cap and a retaining ring can be severed.

With the system according to EP 1 034 810 A1 multiple use can also not be ruled out with certainty.

The elastomer closure elements (tip caps) described in the aforementioned applications for plastic or glass syringes can all be returned to their original conditions after intentional or unintentional opening. Since these closure elements have no tamper-evident seals or indicators, visual recognition of first use is not available. Therefore there is the danger that these elastomer closure elements and thereby the syringe or, respectively, the medication can possibly be used multiple times over, or be mixed up without being noticed.

Intentional or unintentional opening of the tip caps also damages the sterility barrier of the syringe, since the non-sterile environment at the time of opening is in direct contact with the content of the syringe (for example medication). This could allow foreign matter (for example bacteria, germs, viruses, particles, fungi, etc.) to enter the sterile region and to thereby contaminate the content of the syringe. If the contaminated content of the syringe is administered to a patient this could lead to infections, thus endangering the health of the patient.

The original content of a syringe can moreover be altered after opening of the tip cap, and the syringe reclosed without being noticed. Patients could thereby be administered an incorrect dose of a medication, or the content of a syringe could be administered to several patients. This could cause serious health problems.

As described previously, tip caps can—after having been opened—be attached to another syringe with another medication. Traces of one medication could thereby be transferred through the tip cap to another syringe and could thereby contaminate the medication. Because of such contamination the effect of the medication can be altered or destroyed. This may have serious health consequences for patients.

A syringe can be charged after opening of the tip cap with other medications and reclosed without being noticed. The effect of the medication is thus altered. If the altered medication is administered to a patient, this could result in serious health consequences.

With the closure elements according to the current state of the art willful manipulations can occur without being noticed. This represents a great hazard potential for the patient, the user and the pharmacist. In the event of improper use, this detection is not possible due to absent tamper-evidence.

What is needed in the art is a syringe that can avoid some of the disadvantages of the prior art and a closure element to seal a syringe opening that simply and reliably provides single use of the closure cap. Moreover, these functions should be provided with the fewer possible components and associated low manufacturing costs.

### SUMMARY OF THE INVENTION

The objective is met in that a closure element for sealing a distal opening of a syringe cone of a syringe body is provided that overcomes some of the previously described disadvantages.

The syringe closure element includes a fastening element that is arranged or can be arranged on the syringe body around the distal opening, such as around the syringe cone; and a closure cap that closes the distal opening, such as the syringe cone, in a sealing manner and that is connected detachably with the fastening element. The closure cap is connected by material bonding, such as exclusively by material bonding, with the fastening element, which can be a Luer-Lock or Luer-Lock adapter. The material bonded connection can occur through at least one connecting point or at least one connecting surface. The closure cap includes at least one hard region and one elastomer part, the elastomer part tightly sealing the distal opening by simply being placed in a region of a tip of the distal opening. For tight closure, only axially acting forces or an axially acting pressure are essentially or, in other words, predominantly necessary. Forces acting radially upon the tip of the syringe or the distal opening are substantially or completely avoided, so that over the life of the closure element no, or only a minimal increase of the, turning torques occurs.

Due to the fact that the closure cap is connected positively or by material bonding with a Luer-Lock or Luer-Collar or Luer-Lock-adapter by connection points or connection surfaces or, respectively, by weld- and/or adhesion points, a tamper evident closure element is provided in a simple and in a material saving manner that provides visual indication in regard to first use. Multiple uses or improper use can reliably be avoided after breaking open a tamper evident closure element in that the distal opening or, respectively, the syringe cone is not completely surrounded by the elastomer part, but only partially, or is even only placed on the distal opening and the opening closes in an unexpected manner, air tight simply through the placement. A further indication of a multiple use or improper use with an embodiment according to the invention is also ensured by the non-existing thread. After opening or, respectively, after use of the closure element, a positive connection between the closure element and a Luer-Lock or a Luer-Lock-adapter is thereby prevented.

Pressure-tight in this application is understood to be, for example, a tight seal of the distal opening with a pressure of at least 1 bar or more for at least 30 sec.

Due to the fact that the elastomer part only rests on the opening, there is no possibility after breaking open the tamper evident closure element of holding the closure cap, for example, on a thread because of having broken the positive material connection. Also, holding, for example, by gripping the distal opening with the elastomer part, as is the case in the prior art, is not possible. A second use of the closure cap is thus reliably prevented.

In one embodiment, the closure cap is connected with the fastening element on the connection point(s) or connection surface(s) through a joining process. The joining process can be gluing and/or welding, such as laser-welding and/or ultrasonic welding.

The hard region of the closure cap is thus material bonded with the fastening element or, respectively, the Luer-Lock or the Luer-Lock-adapter.

During welding, there are at least one or more contact surfaces on the hard region, for example in the form of welding columns. Local welding points or welding surfaces can thus be produced. During welding, the contact surfaces or connecting points or, respectively, connecting surfaces can be melted through ultrasonic or lateral impingement of the laser beam, thereby producing a materially bonded connection between the hard region of the closure cap and the fastening element. This connection represents the only support on the syringe which is irreparably damaged by opening of the closure element.

Basically, the connecting point(s) or connecting surface(s) can be arranged at different locations of the hard region of the closure cap. The closure cap can include a head part and a foot part, whereby the foot part engages into the fastening element and the head part represents the closure end.

In one embodiment, the connecting point(s) or connecting surface(s) is/are arranged on a side of the head part facing the fastening element. Alternatively, the connecting point(s) or connecting surface(s) can be arranged on a side of the foot part facing the fastening element.

In the case of plastic syringes, the fastening element, the fastening ring, the Luer-Collar, or the Luer-Lock is sprayed onto the injection body, resulting in a single-piece component. In the case of glass syringes, the fastening element, fastening ring, or the Luer-Lock-adapter is a separate component that engages, for example, on a bead of the glass syringe at a predetermined, in other words, predefined height. The tightness of the connection between closure cap and distal opening or, respectively, the cone of the glass syringe is ensured through the predetermined distance and the engagement of the fastening element, fastening ring, or Luer-Lock-adapter.

The syringe closure element according to the current invention can be used for a syringe body consisting of a glass material, as well as one consisting of thermoplastics.

As previously described, the fastening element on a glass syringe body can be a separate component, such as a fastening ring which engages into a bead of the glass syringe at a predetermined height, whereas in contrast on a plastic syringe body—such as one consisting of thermoplastics—the fastening element is part of the syringe body.

In another embodiment, the closure cap includes an elastomer part with which the distal opening, for example the syringe cone, is closed as well as a region that consists of a hard component—a so-called hard region.

The hard component or, respectively, the hard region of the closure cap can be positively interlocked with the elastomer part, such as in a region surrounding the distal opening, for example the syringe cone. The positive interlocking connection of the threaded component or the hard component with the elastomer component offers the advantage that over-tightening of the base component is prevented. In order to design the closure cap to be capable of being sterilized in all conventional sterilization methods, such as gamma radiation, ethylene oxide (ETO), and steam at temperatures of 121° C. and higher, as well as electron radiation, the elastomers of the base material can be selected accordingly. This allows broad use, in addition to glass syringes, with thermoplastic syringes.

The handling region of the closure cap can be integrated into the base material or the base component of the closure cap.

To guarantee protection from multiple uses, another embodiment provides that the base component only surrounds a small part of the syringe cone with the distal opening or even only covers the distal opening. This ensures that the elastomer part cannot hold itself alone on the syringe cone without the positive interlocking connection between hard region and closure cap and the fastening element or, respectively, Luer-Lock or Luer-adapter.

In such a case, the elastomer part has only an axial extension that, at a maximum, is consistent with the axial length of the head part of the closure cap, but it can be even shorter than the axial extension of the head part of the closure cap.

All materials used for the closure cap, in other words the elastomer as well as also the hard components or respectively the hard component, are selected so that they can be used for pharmaceutical applications. The materials can have a USP-class-VI-classification. Moreover, they can also be colored.

Elastomers which are permissible for pharmaceutical applications and butyl rubber can be used.

As hard materials, in particular for the hard component, all permissible thermoplastics can be utilized. Thermoplastics which can survive as many types of sterilization as possible without undergoing significant geometric or material changes can be used. Exemplary thermoplastics are polycarbonate, polyetheretherketone, polyetherimid, polyether block amide, copolyester.

In a method for the production of the syringe closure element, a conventional injection molding process can be used for the hard component, such as the compression or vulcanization method. The elastomer component can moreover also be produced in a transfer-injection or injection molding process. The two components are combined and connected with each other in an additional process step by an assembly system.

The sealing cap for sealing of the distal opening, in particular the syringe closure element, consists of a hard region and an elastomer component. The connection between the elastomer component and the hard component can be positive and can be achieved in various ways.

The various possibilities of a positive connection are discussed in detail below.

In one arrangement of the closure cap, the hard component, which can be a bushing such as an outer cap, includes a through-opening in an axial direction. The opening can be oval, circular or polygonal.

In one arrangement the hard region or, respectively, the hard component, such as a bushing or outer cap, includes at least one axial section which has at least one recess, such as one opening and/or grooves distributed over a circumference of the section.

The distal region of the outside geometry of the elastomer component can be arranged such that it is congruent with the continuous opening of the hard component. In contrast to the hard component or, respectively, the hard region, the distal section of the elastomer component can include one or several radially projecting protrusions or longitudinally shaped ribs which, in design number, correspond to the longitudinally shaped recesses, such as openings inside the hard component or the hard region. In an assembled or fitted condition, the protrusion or ribs of the elastomer component are then accommodated by the openings and/or grooves of the hard component or, respectively, the hard region.

The outside diameter of the ribs or protrusions can be dimensioned so that it is at least as large as the outside diameter of the hard component, such as a little larger. Due to the projection of the ribs or protrusions over the hard component or, respectively, the hard region, an anti-slip surface is created which considerably improves gripping and turning of the closure cap. Additional design elements for this purpose may furthermore be omitted.

Due to the engagement of the ribs or protrusions into the windows, both components are interlocked axially and radially with each other.

By unscrewing the closure cap from the fastening element, the elastomer component is forcibly moved together with the hard component or, respectively, the hard region, in axial and radial direction and is therefore separated from the syringe cone.

In another arrangement of a closure cap, the hard component or, respectively, the hard region includes an opening, such as a continuous stepped opening, in axial direction. The opening of the hard component can be smaller on the distal side than the opening of the hard component on the proximal side. The opening on the proximal side can be ring-shaped and the opening in at least one axial section not circular in its cross section, such as oval or polygonal. In another arrangement with two axial sections of the opening, the opening in the distal region with the non-circular cross section or, respectively, a cross sectional area with non-circular inside contour includes ribs or protrusions which are directed inwards, in other words extending radially from the inside contour into the axial opening of the hard component. The number of ribs or protrusions can correspond with the polygonal arrangement of the opening. In such an arrangement, the distal region of the outside geometry of the elastomer component or the base component is arranged such that it is congruent with the continuous stepped opening of the hard component, meaning that one section of the elastomer component is not circular, in its cross section, and can be polygonal. In one embodiment, the section with the non-circular outside contour include grooves into which the ribs or protrusions of the hard region engage in an assembled state. Due to the engagement of the ribs into the grooves, both components are axially and radially interlocked with each other.

By unscrewing the closure cap from the fastening element, the elastomer component is forcibly moved together with the hard component in axial and radial direction and is therefore separated from the syringe cone.

In yet another arrangement of a connection of hard component or, respectively, hard region and elastomer component, the hard component includes an opening, such as a continuous stepped opening in axial direction. The openings at the ends of the hard component in axial direction are larger than that of an opening located between them. In other words, the cross section of the intermediate opening in axial direction has a first cross section (Q₁) which is always smaller than the second cross section (Q₂) and the third cross section (Q₃) of the end regions of the opening in axial direction. The inside contour of the opening at the ends can be circular, oval or polygonal. The inside contour of the opening in the first axial region is not circular or round, such as polygonal, multi-edged, or oval. The elastomer component or, respectively, the elastomer part is designed so that it is congruent with the continuous stepped opening of the hard component or, respectively, hard region, at least in the distal region. This type of arrangement provides an axial interlocking of hard and elastomer component or, respectively, base component.

By unscrewing the closure cap from the fastening element, the elastomer component or, respectively, elastomer part is forcibly moved together with the hard component or, respectively, the hard region in axial direction and is therefore separated from the syringe cone. Due to the non-circular, which can be polygonal, arrangement of the two components, rotational security is achieved. This means, if the hard component or, respectively, the hard region is turned to be released, the elastomer component or, respectively, the elastomer part turns with it. Moreover, due to the polygonal design, the torque when unscrewing the closure cap is reduced since the elastomer part turns along with it. The expenditure of force can thus be greatly reduced.

The technical advantages of the previously described arrangements of the closure cap are found in an efficient and robust design. The axial and radial connection of both components can be realized in a simple manner. This in turn results in that both components can be produced cost effectively and in that the expenditure for the production can be kept low.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIGS. 1A-1C, 4A-4C, and 7A-7C illustrate a first embodiment of a syringe closure element with connecting points on the head part of the hard region of the closure cap, whereby the syringe is a glass syringe and the fastening element is a separate component with which the closure cap is positively connected;

FIGS. 2A-2C, FIGS. 5A-5C, and FIGS. 8A-8C illustrate another embodiment of a syringe closure element with connecting points on the head part of the hard region of the closure cap, whereby the syringe is a plastic syringe and the fastening element with which the closure cap is positively connected is part of the plastic syringe;

FIG. 2D illustrates a pressure-tight seal of the distal opening with an embodiment of an elastomer part according to the present invention;

FIG. 2E illustrates a pressure-tight seal of the distal opening with the elastomer part according to the prior art;

FIGS. 3A1-3A3, FIGS. 6A1-6A3, and FIGS. 9A1-9A3 illustrate a hard region of a closure cap for a closure element according to FIGS. 1A-2C, FIGS. 4A-5C, and FIGS. 7A-8C;

FIGS. 3B1-3C3, FIGS. 6B1-6C3, and FIGS. 9B1-9C3 illustrate an elastomer part of a closure cap for a closure element according to FIGS. 1A-2C, FIGS. 6B1-6C3, FIGS. 4A-5C, and FIGS. 7A-8C;

FIGS. 3D1-3F, FIGS. 6A-6F, and FIGS. 9D1-9F illustrate a closure cap consisting of an elastomer part and hard region for a closure element according to FIGS. 1A-2C, FIGS. 4A-5C, and FIGS. 7A-8C;

FIGS. 10A-10C, FIGS. 13A-13C, and FIGS. 16A-16C illustrate another embodiment of a syringe closure element with connecting points on the foot part of the hard region of the closure cap, whereby the syringe is a glass syringe and the fastening element is a separate component with which the closure cap is positively connected;

FIG. 10D is a top view of an embodiment of a fastening element according to the present invention;

FIG. 10E is a cross-section through the fastening element shown in FIG. 10D along line A-A, with connecting points or connecting surfaces;

FIGS. 11A-11C, FIGS. 14A-14C, and FIGS. 17A-17C illustrate another embodiment of a syringe closure element with connecting points on the foot part of the hard region of the closure cap, whereby the syringe is a plastic syringe and the fastening element with which the closure cap is positively connected is part of the plastic syringe;

FIGS. 12A1-12A3, FIGS. 15A1-15A3, and 18A1-18A3 illustrate a hard region of a closure cap for a closure element according to FIGS. 10A-11C, FIGS. 13A-14C, and FIGS. 16A-17C;

FIGS. 13A-14C, FIGS. 16A-17C, FIGS. 12B1-12C3, FIGS. 15B1-15C3, and FIGS. 18B1-18C3 illustrate an elastomer part of a closure cap for a closure element according to FIGS. 10A to 11C, FIGS. 13A-14C, and FIGS. 16A to 17C; and

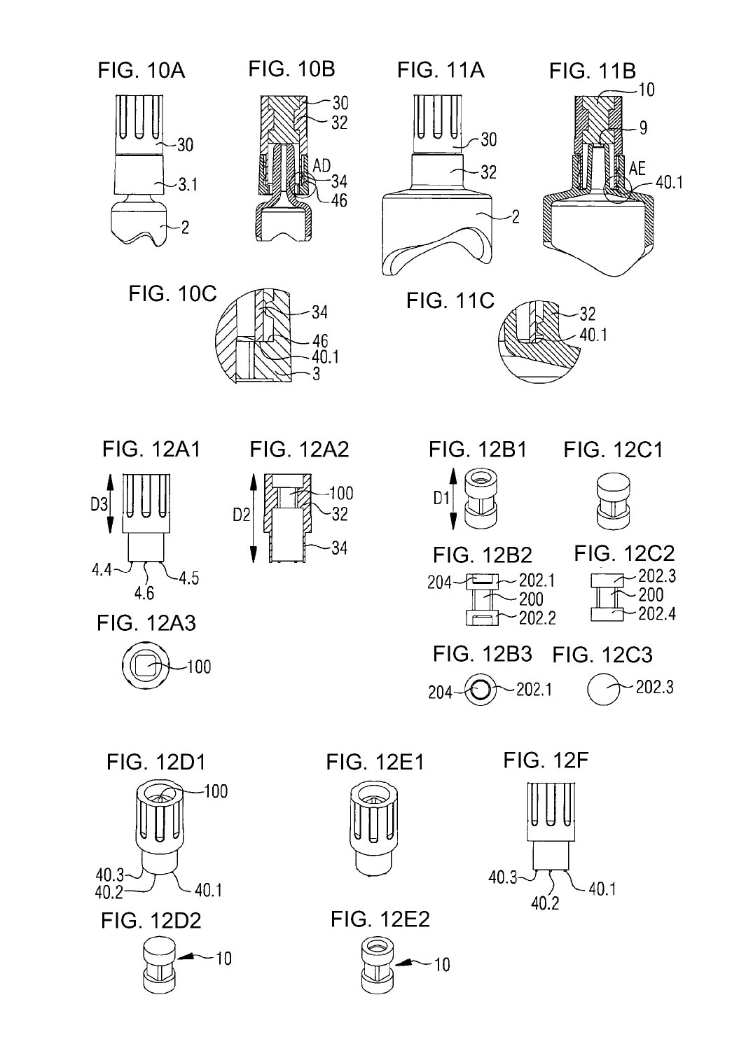

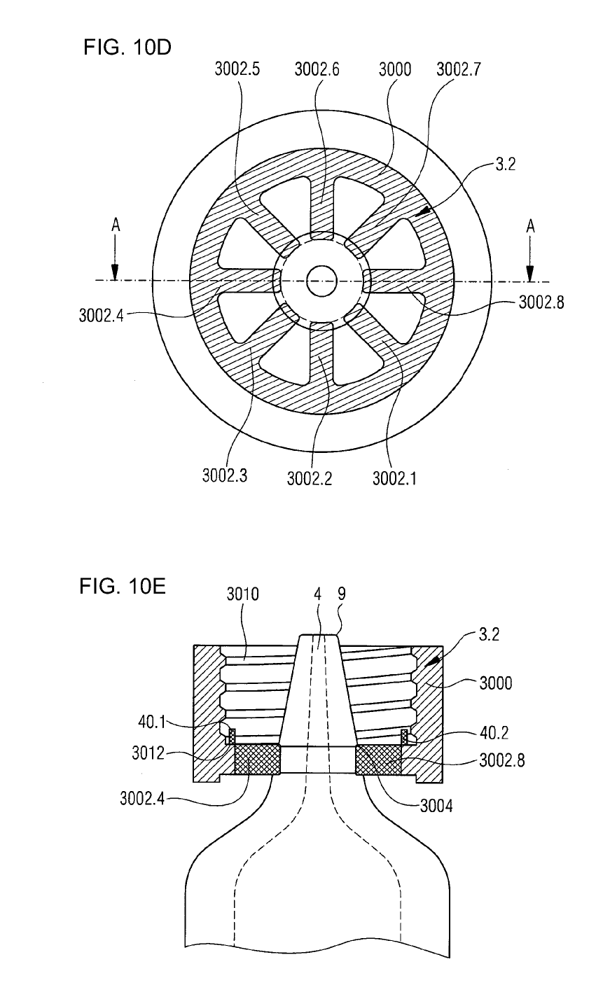

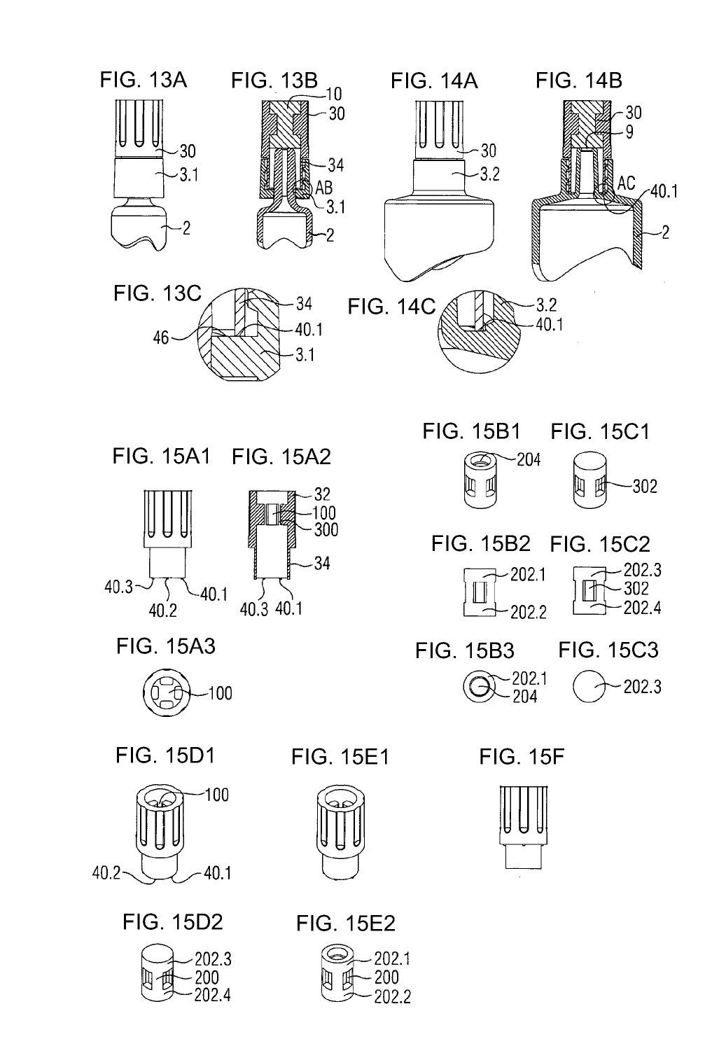

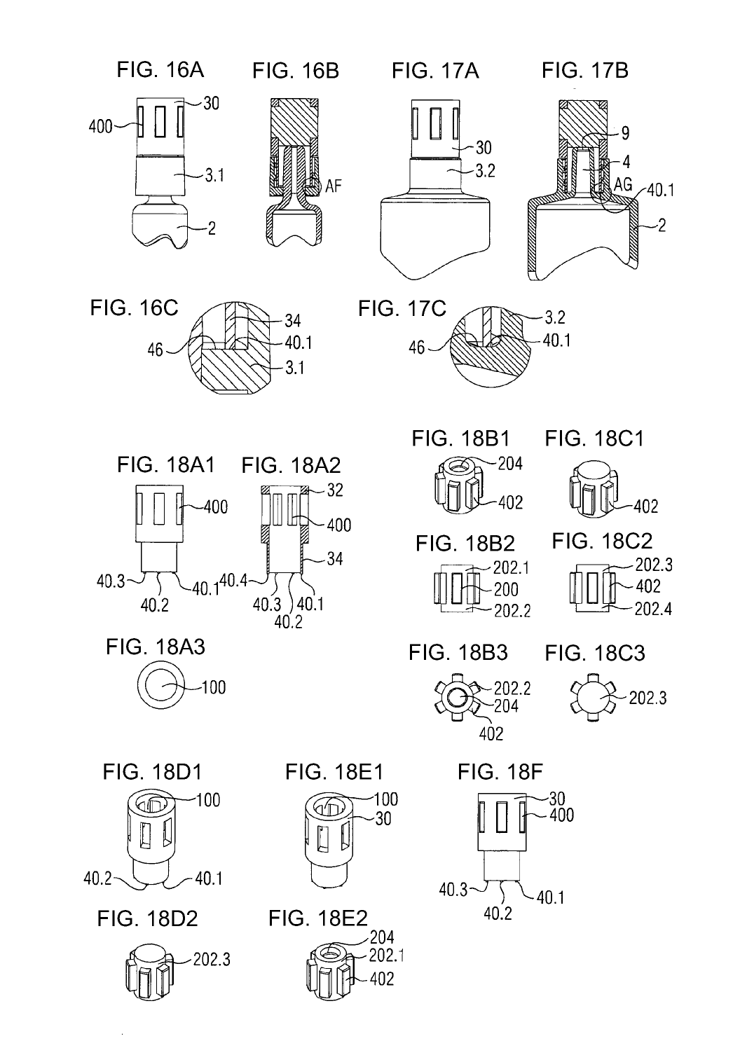

FIGS. 12D1-12F, FIGS. 15D1-15F, and FIGS. 18D1-18F illustrate a closure cap, consisting of an elastomer part and hard region according to FIGS. 10A-11C, FIGS. 13A-14C, and FIGS. 16A-17C.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

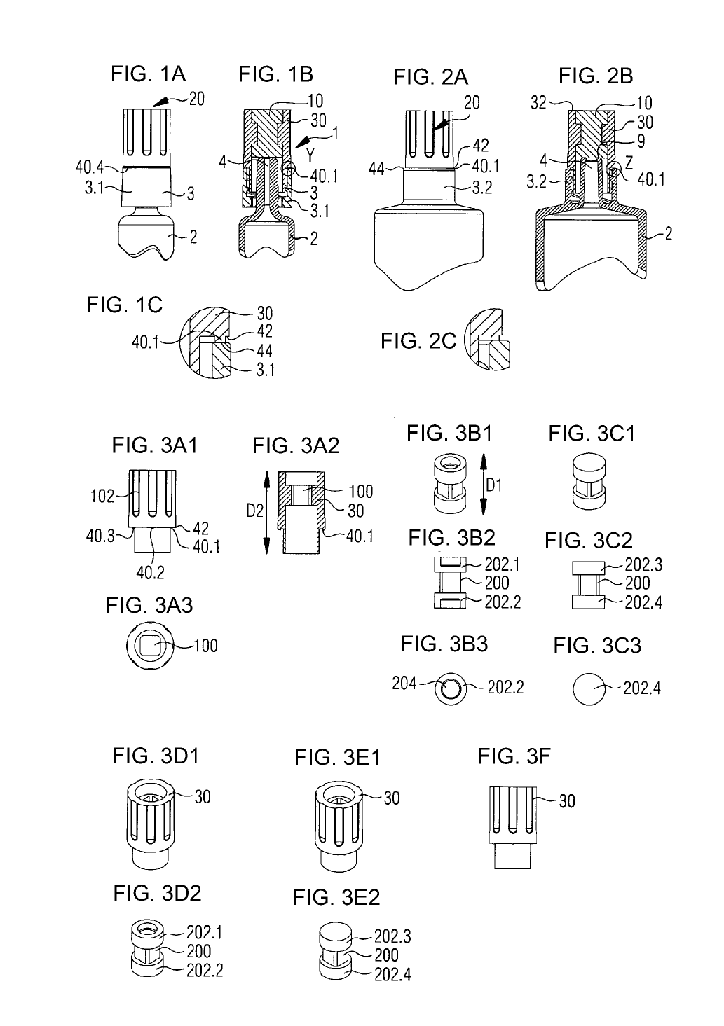

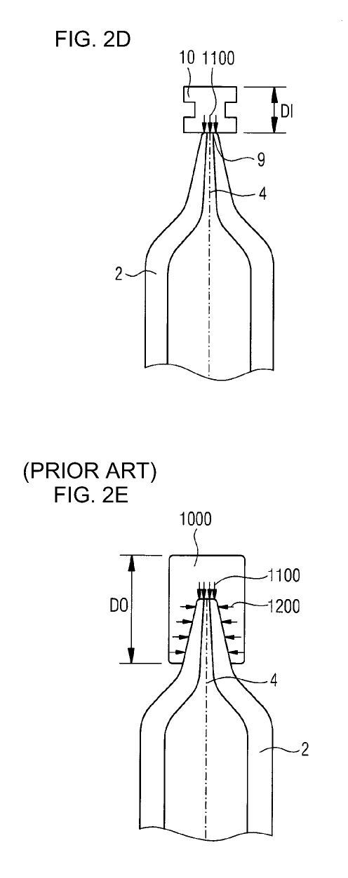

An embodiment of a complete syringe closure element 1 according to the invention is illustrated in FIG. 1A-2C, 4A-5C, 7A-8C. FIGS. 1A-1C, FIGS. 4A-4C, FIGS. 7A-7C illustrate hereby one embodiment of the present invention, wherein syringe body 2 is a glass syringe and fastening element 3 is a separate component, a so-called Luer-Lock-adapter 3.1. FIGS. 2A-2C, FIGS. 5A-5C, and FIGS. 8A-8C illustrate one embodiment of the invention, wherein syringe body 2 is a plastic syringe and fastening element 3, the so-called Luer-Lock 3.2 or, respectively, Luer-Lock collar is part of syringe body 2. Complete syringe closure element 1 comprises a syringe body 2 with a distal opening 4. On distal opening 4, elastomer part 10 of closure cap 20 is placed, which can comprise an elastomer that is approved for pharmaceutical use. Distal opening 4 of syringe body 2 is hereby closed and sealed. In contrast to the prior art, elastomer part 10 encloses the distal opening 4 or, respectively, the syringe cone only partially or not at all when resting on it, so that the closure element 1 after first use can no longer be fastened on the syringe 2, since there is no longer a positive connection possible on the closure element 1. The support surface for the elastomer part can then as shown in FIG. 2B—be only edge 9 of distal opening 4 of the syringe cone. “Pressure-tight” in this application is understood to be, for example, a tight seal or closure of the distal opening at a pressure of at least 1 bar or more for at least 30 seconds. Thermoplastic elastomers and butyl rubbers are acceptable elastomers.

The elastomers are selected such that the entire closure cap 20, comprising a hard region 30 into which elastomer part 10 is introduced, is capable of being sterilized in all conventional sterilization methods, for example gamma radiation, at for example 20 KGy or ETO or steam at temperatures for example of 121° C. and higher for at least 20 min., as well as electron radiation. This allows broad use of closure caps 20 in glass syringes as well as thermoplastic syringes. In the case of plastic syringes, the fastening element or, respectively, fastening ring 3 and syringe body 2 of the plastic syringe can be a single component, as shown.

In addition to elastomer part 10, closure cap 20 includes at least one hard part or component 30 as a second component.

The hard component or, respectively, hard region 30 is material-bonded with fastening element 3, in other words with Luer-Lock 3.1 and in the case of a plastic syringe with Luer-Lock-adapter 3.2—such as by shaping technology

In one embodiment, illustrated in FIGS. 1A to 9C3, contact points or contact surfaces 40.1 can be provided on underside 42 of hard region 30 of the closure cap 20 which is located opposite top side 44 of fastening element 3 or, respectively, 3.1 or 3.2. The attachment points or, respectively, contact surfaces can be, for example, welding columns which are melted, for example, through ultrasonic or lateral coupling of laser radiation onto the welding surfaces, thereby providing a materially bonded connection between underside 42 of hard region 30 and top side 44 of the fastening element 3 and, consequently, a material-bonded connection between closure cap 20 and fastening element 3. This connection represents the only holding device on the syringe and is irreparably damaged by removal of the closure cap 20 or, respectively, tip caps. The connecting surface or, respectively, the welding columns 40.1 is illustrated especially detailed in FIGS. 1C, 2C, 4C, 5C, 7C and 8C. In one embodiment of the invention, each of these FIGS. illustrates that front surface 42 of the hard region is connected by material bonding through the connecting points, or, respectively, connecting surfaces 40.2 with top side 44 of the fastening element, or respectively, through Luer-Lock or Luer-Lock-adapter, in the case of glass syringes, materially bonded through a joining process such as, for example, adhesive bonding, ultrasonic welding or laser welding.

Alternatively to the illustrated connection between the end face of hard region 30 and top side 44 of fastening element 3, as shown in FIGS. 1A to 9C3, it is possible according to another embodiment—as illustrated in FIGS. 10A to 18C3—to not connect the hard region 30 of closure cap 20 which consists of a head part 32 and a foot part 34 in the region of head part 32 as is the case in the arrangement shown in FIGS. 1A to 9C3, but instead to connect foot part 34 of hard region 30 of the closure cap with base 46 of fastening element 3 in the embodiment of Luer-Lock 3.1 or respectively Luer-Lock-adapter 3.2. This other embodiment, wherein the connecting points or, respectively, connecting surfaces of hard region 30 are provided with fastening element 3 in the region of foot part 34 of hard region 30 of the closure cap, is illustrated in FIGS. 10A to 18C3. This connection can be seen in particular in FIGS. 10C, 11C, 13C, 14C, 16C and 17C. Same components as in FIGS. 1A to 9C3 are illustrated for the other embodiment in FIGS. 10A-18C3.

In FIG. 10D, the fastening element 3, representative of that shown in FIGS. 11A-11C, 14A-14C and 17A-17C is shown in a top view in further detail. Fastening element 3.2 illustrated in FIG. 10D is a Luer-Lock-adapter as is generally used with glass syringes. In the illustrated arrangement, the Luer-Lock-adapter surrounding distal opening 4 comprises an annular tube 3000, wherein grippers 3002.1, 3002.2, 3002.3, 3002.4, 3002.5, 3002.6, 3002.7, 3002.8 extend from annular tube 3000 radially toward the inside to the distal opening 4. Grippers 3002.1, 3002.2, 3002.3, 3002.4, 3002.5, 3002.6, 3002.7, 3002.8, which are under tension, engage into an indentation around the distal opening and thus, through clamping, secure the position of the Luer-Lock-adapter on distal opening 4 of the glass syringe. Annular tube 3000 is largely tension free while the grippers are under tension. FIG. 10E illustrates a cross-section along line A-A in FIG. 10D. Grippers 3002.4, 3002.8 that under tension fit against the glass syringe in the region of indentation 3004 are clearly recognizable. Also clearly recognizable is tube 3000 which comprises a thread 3010 for screwing on a needle after breaking open the closure element, sealing the distal opening, as well as a support surface 3012 or, respectively, base 46 which accommodate connecting points 40.1, 40.2 of the non-illustrated hard region of the closure cap and provide for material-bonded connection of the closure cap with the fastening element. The material bonded connection of closure cap and fastening element is arranged in the region of the fastening element that is not under pressure, so that an unintended break of the material-bonded connection of the regions that are under pressure, for example during and after the assembly of the fastening element onto distal opening 4 of the glass syringe, is reliably prevented.

As shown in FIG. 10E, connecting points 40.1, 40.2 are arranged in regions where there is little to no mechanical tension—such as during or after the assembly of the Luer-Lock-adapter or, respectively, the fastening element on a glass syringe. These regions are located in the region of tube 3000 or, respectively, the support surface 3012, for example between two grippers 3002.4, 3002.5 or 2002.1, 2002.8 that secure the Luer-Lock-adapter positively with the glass syringe.

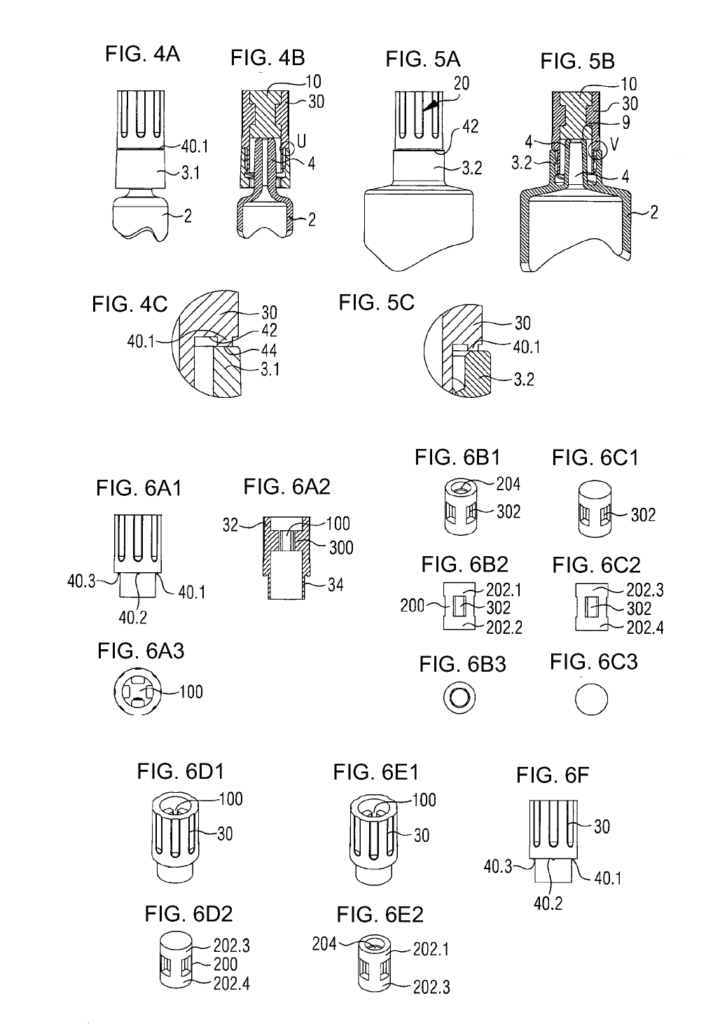

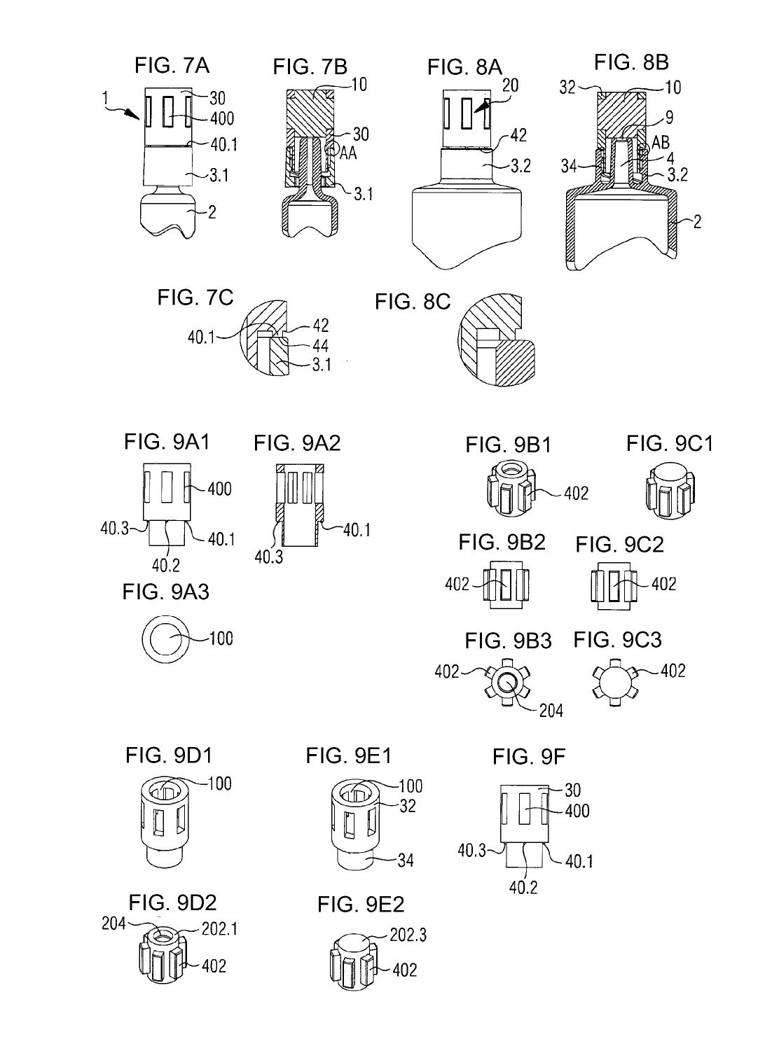

Various arrangements of closure cap 20 are illustrated in FIGS. 3A1 to 3F, FIGS. 6A1 to 6F, as well as FIGS. 9A1 to 9F. Here, FIGS. 3A1 to 3A3, FIGS. 6A1 to 6A3 and FIGS. 9A1 to 9A3 show different versions of hard region 30 of closure cap 20. Clearly recognizable in the arrangement according to FIG. 3A1 to 3A3 is the opening in axial direction 100 that is introduced into hard region 30 into which the elastomer part illustrated in FIGS. 3B1 to 3C3 can be inserted. Clearly visible in FIGS. 3A1 to 3A3 are connecting points 40.1, 40.2, 40.3 that are arranged on underside 42 of the head part and that can be connected by means of a joining process through material bonding with fastening element 3. Clearly recognizable is also the foot part hard region 30. In the arrangement shown in FIG. 3A1 to 3A3, the hard region includes grip recesses 102 for gripping the closure cap. The elastomer part illustrated in FIGS. 3A1 to 3A3 that is inserted as illustrated in FIGS. 3D1 to 3D2 into the hard region according to FIGS. 3A1 to 3A3 includes two variations. The elastomer part can be designed symmetrically, allowing for very easy manufacture. The elastomer part can include a center part 200 and a head part 202.1, 202.2. Head part 202.1, 202.2 can include an indentation 204 into which the syringe cone can engage in a closed state of the closure cap. The indentation can be such that only a small part of the syringe cone is surrounded. In the arrangement shown in FIG. 3C1, end part 202.3, 202.4 is designed without indentation so that—as shown in FIG. 2B—the elastomer part with sealed syringe body only rests on the syringe cone.

An embodiment of the closure element according to the present invention is illustrated in FIG. 2D. FIG. 2D shows symmetrical elastomer part 10 resting on edge 9 or, respectively, the tip of distal opening 4. FIG. 2E, in contrast, shows a prior art arrangement wherein elastomer part 1000 completely surrounds distal opening 4. In the embodiment according to the present invention shown in FIG. 2D, elastomer part 10 rests on distal opening 4 after assembly of the closure cap with hard region (not illustrated). An axial pressure 1100, only sufficient for tightness, is exerted upon the distal opening 4. The extension of elastomer part 10 with D1 compared to elastomer part 1000 according to the prior art with D0 is substantially shorter, so that in contrast to the prior art a radial pressure 1200 in addition to the axial pressure 1100 is not exerted upon distal opening 4. In contrast to the prior art, the additional radial pressure is not necessary for the tightness of the distal opening 4. A radial pressure that is too great, such as occurs in the prior art with nearly complete surround of the distal opening, is disadvantageous in regard to the mechanical characteristics, since with increasing dwell time of elastomer part 10 on distal opening 4 of the syringe the value of the turning torque increases in the prior art. In the prior art, this is attributed to the fact that the elastomer sets with increasing time on the tip, so that toward the end of the serviceable life of the product, turning or removal of the elastomer part is considerably more difficult. According to the present invention, only the tip or, respectively, edge 9 of distal opening 4 of syringe body 2 is sealed. Because of the mostly or, respectively, completely absent radial forces 1200 an increase in the turning torques can be avoided; in other words, an additional radial pressure, as is common in the prior art, is absent.

As is clearly recognizable, in an assembled state the elastomer part is substantially shorter in its axial extension D1 than the entire hard component 30 with extension D2 or, respectively, than head part 32 of the hard component with extension D3.

The arrangement according to FIGS. 6A1 to 6F shows essentially a difference in the design in the shape of the elastomer, as illustrated in FIGS. 6B1-6C3. Here too, the elastomer part 6b.1 -6c.3 is designed substantially symmetrical and is considerably shorter in its length than entire hard region 30 with length D2. Elastomer part 10 is carried along in the rotational movement by hard component 30 in that protrusions 300 in the hard component 30, as shown in FIGS. 6a.1 to 6a.3, engage into indentation 302 of the elastomer part, as illustrated in FIGS. 6D1 to 6C3. Same components as in previous Figs. are identified with the same reference numbers. Protrusions 302 are again located in center part 200 of the elastomer component. End surfaces 202.1 202.2 of the elastomer part are equipped with indentations 204, as is the case in the embodiment shown in FIGS. 3B2 and 3B2, whereas in contrast the end surfaces according to FIGS. 6C1 and 6C2 are flat.

The embodiments shown in FIGS. 9A1 to 9F essentially differ from the preceding arrangements of a closure cap in that hard region 30 has openings 400. The corresponding elastomer part is illustrated in FIGS. 9B1 to 9C3 and also has a symmetrical shape as well as protrusions 402 that engage into the windows of hard region 400 and protrude beyond same, so that the closure cap can be gripped positively in the head region. The elastomer part is carried along during turning due to protrusions 402 of the elastomer part that engage into windows 400 or respectively into the longitudinal cut-outs in the hard region.

Same components as in previous Figs. are again identified with the same reference numbers.

The Figs. of another embodiment of the invention differs basically in that the connecting points are not arranged on head part 32 of hard region 30, but instead on foot part 34 of the hard region. Other than that, the embodiments according to FIGS. 12A1 to 12B2 correspond with those according to FIGS. 3A1 to 3F. Accordingly, same components as shown in FIGS. 3A1 to 3F are identified with the same reference numbers in FIGS. 12A1 to 12F.

Reference should again be made to connecting points 40.4, 40.5, 40.6 that are arranged on foot part 45 of hard region 30.

With the exception of the configuration of connecting points 40.4, 40.5, 40.6 in foot region 34, the embodiment according to FIGS. 15A1 to 15F corresponds with that according to FIGS. 6A1 to 6F. In this respect, reference is made to that description and those reference numbers.

FIGS. 18A1 to 18F correspond essentially to the embodiment of the closure cap according to FIGS. 9A1 to 9F. In this respect, reference is made to that and same reference numbers are used. The only difference in the embodiment according to FIGS. 18A1 to 18F compared with the embodiment in FIGS. 9A1 to 9F is the configuration of connecting points 4.4, 4.5, 4.6 in foot region 34 of hard region 30.

With the closure element according to the invention, a closure element for a syringe cone is specified in that it can reliably be used only once and in that any possibility of a second use is prevented in a simple manner. This is ensured in that a material-bonded connection is provided between the closure cap and the fastening element on the syringe cone which must be broken during use. Due to the design of the elastomer part that, surprisingly, also provides a tight closure of the syringe body when only resting on the syringe cone and not, or only partially, surrounding same, ensures that the closure element after having broken the material-bonded connection after a first use can no longer be secured on the syringe cone. The hard region has no thread. A positive locking connection is therefore impossible.

Moreover, simple manufacturability is ensured since the elastomer part is substantially symmetrical. An additional advantage of the symmetrical design is that the elastomer parts do not need to be aligned in a certain direction. For the elastomer parts, expensive sorting devices have been necessary to date in the prior art in order to align the elastomer parts in a certain assembly direction. This alignment is expensive and can be avoided with the current invention.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A closure element for sealing a distal opening formed in a syringe cone of a syringe body, comprising:
a fastening element that one of is arranged and is configured to be arranged on said syringe body around said distal opening; and
a closure cap that closes said distal opening in a sealing manner and that is connected detachably with said fastening element, said closure cap comprising at least one hard region and at least one elastomer part, said closure cap being connected in a region of said fastening element by material bonding with at least one connecting point or at least one connecting surface of said at least one hard region, wherein said at least one elastomer part tightly seals said distal opening essentially through axial pressure by being placed one of on and surrounding a short section in a region of a tip of said distal opening.

2. The closure element according to claim 1, wherein said closure cap is connected with said fastening element on said at least one connection point or at least one connection surface through a joining process.

3. The closure element according to claim 1, wherein said at least one hard region of said closure cap includes a head part and a foot part, and said at least one connecting point or said at least one connecting surface is arranged on a side of said head part facing said fastening element.

4. The closure element according to claim 1, wherein said at least one hard region of said closure cap includes a head part and a foot part, and said at least one connecting point or said at least one connecting surface is arranged on a side of said foot part facing said fastening element.

5. The closure element according to claim 1, wherein said at least one connecting point or said at least one connecting surface is formed as a welding column melted at least partially due to effects of energy so that a materially bonded connection is established with said fastening element.

6. The closure element according to claim 1, wherein said syringe body consists of at least one of a glass material and a thermoplastics material.

7. The closure element according to claim 1, wherein said at least one hard region of said closure cap comprises a head part and a foot part and defines an axial length, said at least one elastomer part having an axial extension that at a maximum is consistent with said axial length of said head part of said at least one hard region.

8. The closure element according to claim 1, wherein said at least one elastomer part is configured such that it surrounds only a short section of said distal opening of said syringe cone or envelopes said syringe cone only partially in such a manner that said at least one elastomer part is held only together with said at least one hard region on said distal opening of said syringe cone, or only rests on one of an edge and said tip of said distal opening of said syringe cone.

9. The closure element according to claim 1, wherein said at least one elastomer part comprises a thermoplastic elastomer that is approved for pharmaceutical applications.

10. A closure element according to claim 1, wherein said at least one hard region of said closure cap includes at least one axial section which comprises at least one recess and said at least one elastomer part includes an additional axial section that comprises at least one of at least one radially projecting protrusion and at least one rib distributed over a circumference of said additional axial section and that are arranged such that said at least one of said at least one protrusion and said at least one rib of said at least one elastomer part is accommodated by said at least one recess in an assembled state of said closure cap.

11. The closure element according to claim 10, wherein said at least one hard region is a bushing, wherein said bushing has an opening in an axial direction to accommodate said at least one elastomer part in an assembled state of said closure cap.

12. The closure element according to claim 11, wherein said at least one recess is a substantially longitudinal opening in said bushing.

13. The closure element according to claim 12, wherein said at least one elastomer part includes at least one protrusion, said at least one protrusion projecting through said at least one recess and protruding over a circumference of said bushing.

14. The closure element according to claim 11, wherein said opening in said axial direction of said bushing has a cross section that is one of circular, oval, and polygonal.

15. The closure element according to claim 1, wherein said at least one hard region is a bushing including an opening in an axial direction to accommodate said at least one elastomer part in an assembled state of said closure cap, wherein said opening has a cross-sectional surface in at least one axial section that has an inside contour that is not circular.

16. The closure element according to claim 15, wherein said at least one elastomer part has a first axial section and a second axial section in an axial direction, said first axial section comprising a first cross-sectional surface and said second axial section comprising a second cross-sectional surface, wherein at least said first cross-sectional surface has an outside that corresponds to said inside contour of said opening in said axial direction of said bushing in said axial section in which said cross section of said opening is not circular.

17. The closure element according to claim 16, wherein said first axial section of said at least one elastomer part has at least one recess, at least one protrusion of said bushing that projects beyond said inside contour of said opening of said bushing engaging into said at least one recess of said at least one elastomer part.

18. The closure element according to claim 14, wherein said at least one elastomer part has a third axial section comprising a third cross-sectional surface.

19. The closure element according to claim 18, wherein at least one of said second cross-sectional surface and said third cross-sectional surface is larger than said first cross-sectional surface.
